(19) United States
(12) Patent Application Publication
(10) Pub. No.: US 2021/0346549 A1
(43) Pub. Date: Nov. 11, 2021

(54) Non-Contact Sanitizing Products and Elements

(71) Applicant: Segan Industries, Inc., Burlingame, CA (US)

(72) Inventors: Hans O. Ribi, Hillsborough, CA (US); Mikaela K. Ribi, Hillsborough, CA (US)

(21) Appl. No.: 17/210,960

(22) Filed: Mar. 24, 2021

Related U.S. Application Data
(60) Provisional application No. 62/994,743, filed on Mar. 25, 2020.

(51) Int. Cl. A61L 2/18; A61L 2/26; B65D 33/25

(52) U.S. Cl. CPC A61L 2/18; A61L 2101/34; B65D 33/25; A61L 2/26

(57) ABSTRACT

The invention describes a simple to use, affordable, sustainable, and safe decontamination product that combines a sealed container that efficiently decontaminates the surface of a potentially contaminated product placed in the container. Upon removal of the potentially exposed article from the container, the volatile disinfectant evaporates from the surface of the article rendering the article in full compliance for use.

### CROSS-REFERENCE

Pursuant to 35 U.S.C. § 119 (e), this application claims priority to the filing date of United States Provisional Patent Application Serial No. 62/994,743 filed Mar. 25, 2020, the disclosure of which application is incorporated herein by reference in its entirety.

### INTRODUCTION

Coronavirus disease and other infectious disease are caused by destructive pathogens. The disease causes respiratory illness (like the flu) with symptoms such as a cough, fever, and in more severe cases, difficulty breathing. Protective actions include washing hands frequently, avoidance of face touching and avoidance of dose contact (1 meter or 3 feet) with people who are unwell. Mass epidemic and pandemic episodes such as COVID-19 are devastating to society. Any practical means to prevent and mitigate transmission have become exceedingly important. Likewise, any products or opportunities that reduce transmission of other viral and bacterial pathogens can be equally applicable.

Importantly, virus and pathogens including, but not limited to, SARS-CoV-2 (the virus responsible for COVID-19 disease) spread primarily through contact with an infected person when they cough or sneeze. Such viruses also spread when a person touches a surface or object that has the virus on it, following which the person then touches their eyes, nose, or mouth.

### SUMMARY

The invention involves a sealed container (e.g. plastic bag or the like), that will include products purchased from a retail, on-line store, commercial and/or industrial company and is a simple/safe means (drop-in or integrated) to kill viral and bacterial contagions that in any way would be associated with products purchased and intended for the public, private, or elected sector distribution. Aspects of the invention include, but are not limited to, a container/decontaminating element that saturates the container and articles contained within the container. The decontamination element within the container saturates the container with a vapor at a level of an agent that kills contagions to maintain safety at a reasonable or greater level.

The invention involves a sealed container, bag, box or otherwise that can contain products, items, or other article that may have a surface potentially exposed to a viral or bacterial pathogens. The article may have been purchased from a retail, on-line store, commercial and/or industrial company and would be delivered to a location. The article may have been surface contaminated at a location. The invention provides for a practical, affordable, and safe means to non-destructively expose the article to a vaporized antiseptic, sterilization, sanitizer or other volatile disinfectant to kill the viral or bacterial pathogen. The invention avoids submersing or spraying the article so that the article's integrity is maintained. Upon removal of the potentially exposed article from the sealed container, the volatile disinfectant will evaporate from the surface of the article rendering it in full compliance for use.

The invention offers a plurality of safety, sanitization, ease of use benefits and may enjoy wide application by avoiding submersing or spraying the contained article thereby maintaining the article's integrity. Upon removal of the potentially exposed article from the sealed container, the volatile disinfectant will evaporate from the surface of the article rendering it in full compliance for use.

Articles that may treated with devices of the invention may vary, and may be any consumer, food-service, health care, residential, hospital, communal, public, private, medical, safety, toy, commercial product, industrial product, component, reading material, clothing item, jewelry, or any other peripheral item that is subject to human or animal contact leading to potential transmission to another being.

Structural elements described herein prevent the contamination of product features that come in contact by touching and benefit by preventing the transfer of contaminating matter, micro-organisms, bacteria, viruses, dirt, chemicals, or other substances. There is an on-going need to improve the performance, public exposure and sanitation, packaging, ease-of use, and differentiation of such products. Product improvements at affordable costs are increasing in demand.

The invention includes, but is not limited to, a container/decontaminating element that saturates the container and articles contained within the container. The decontamination element within the container saturates the container with a vapor at a level of an agent that kills contagions so as to keep the articles safe at a reasonable or greater level.

Public safety and sanitation is a major issue in food and other services. Pathogen transfer from table, counter tops, consumer products, industrial products, commercial and medical products, health care products, food serving surfaces can be sigificantly mitigated utilizing the invention described below. Furthermore, reduction in food transfer from a serving utensil to a surface can be equally reduced. Further, shape directed elements directly integrate both sanitary and ease-of-use features into the products containing them. Elements can be readily incorporated into high-volume consumable products to increase utility, function and features of the consumable product at minimal incremental cost and adjustment to production and manufacturing processes.

The invention further reports processes and compositions that enable products with differentiating features which product would otherwise be deficient for their intended use and application.

### BRIEF DESCRIPTION OF THE FIGURES

FIGS. 1 A and 1B provide views of the construct and removal of the laminating barrier for use.

FIGS. 2A and 2B provide views of a construct according to another embodiment of the invention.

FIGS. 3A and 3B provide views of a sanitizing device according to another embodiment of the invention.

FIGS. 4A and 4B provide views of a sanitizing device according to another embodiment of the invention.

FIGS. 5A and 5B provide views of a sanitizing according to another embodiment of the invention.

### DETAILED DESCRIPTION

The invention involves the incorporation and enablement of multiple interactive elements into high-volume consumable products to increase utility, function and features of the consumable product at minimal incremental cost and adjustment to production and manufacturing processes. The invention further reports processes and compositions that enable consumable products with differentiating features which product would otherwise be deficient for their intended use and application.

The invention avoids submersing or spraying an article so that the article's integrity is maintained. Upon removal of the potentially exposed article from the sealed container, the volatile disinfectant will evaporate from the surface of the article rendering it in full compliance for use. Articles are may be any consumer, food-service, health care, residential, hospital, communal, public, private, medical, safety, toy, commercial product, industrial product, component, reading material, clothing item, jewelry, or all other peripheral item that is subject to human or animal contact leading to potential transmission to another being. Aspects of the invention further include methods of making and using the sanitization and/or decontaminating vessel with application to a wide range of articles and/or consumer products.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

The invention a plurality of safety, sanitization, ease of use and wide application by avoiding submersing or spraying the contained article thereby maintaining the article's integrity. Upon removal of the potentially exposed article from the sealed container, the volatile disinfectant will evaporate from the surface of the article rendering it in full compliance for use.

Articles may be any consumer, food-service, health care, residential, hospital, communal, public, private, medical, safety, toy, commercial product, industrial product, component, reading material, clothing item, jewelry, or all other peripheral item that is subject to human or animal contact leading to potential transmission to another being.

Herein we describe the incorporation and enablement of sanitizing elements into high-volume consumables products to increase utility, function and features of the product at an affordable cost and adjustment to production and manufacturing processes. The invention further reports processes and compositions that enable sanitizing vessel products with differentiating features which products would otherwise be deficient for their intended use and application.

Structural Plastics and Resins for Containment and/or Support:

Bag material compositions can include but are not limited to polypropylene, polyethylene, related polyolefin types, cellophane, nylon, laminates and co-extruded plastic types, silicone, woven types that are air-tight. Examples below illustrate the diversity of plastic choices depending on the application not intended as limitations for their use as sanitizing containers.

Poly-lactic acid (PLA) and compounded CPL a can find use as a commercially compostable version for an incremental benefit of environmental friendliness. Other bio-degradable bags, such as those from starch. Starch, obtained from corn or potatoes, can be converted into lactic acid, which can be polymerized to the biodegradable plastic known as polylactide. Another solution is to add an ultraviolet-light absorber to make the material degrade when exposed to sunlight. Unfortunately, these solutions can make the plastics more expensive, so they haven't caught on with consumers.

Plastic bags typically are made from one of three basic types: high-density polyethylene (HDPE), low-density polyethylene (LDPE), or linear low-density polyethylene (LLDPE). Those thick, glossy shopping bags from the mall are LLDPE, while grocery bags are HDPE, and garment bags from the dry cleaner are LDPE. The major difference between these three materials is the degree of branching of the polymer chain. HDPE and LLDPE are composed of linear, unbranched chains, while LDPE chains are branched.

Branching can influence a number of physical properties including tensile strength and crystallinity. The more branched a molecule is, the lower is its tensile strength and crystallinity. Garment back thicknesses are insufficient due to weakness and flimsyness. They are made from highly branched LDPE.

High-Density Polyethylene—HDPE: High-Density Polyethylene is one of the most common types of plastic bags. HDPE is used in the manufacture of papers for use in shopping outlets. The chemical composition of the polymer that forms HDPE features a long chain of straight molecules whose structure is linear from the beginning to the end. This polymer features minimal branching. This chemical structure results in highly dense plastic bags that are sturdy, light in weight and relatively opaque. The preference of plastic bags made of HDPE lies in their ability to hold a lot of items or weight without breaking. The bag is also extremely resistant to chemicals, water, and even heat. It can conceal the content carried because of its opaque nature. Further, it is safe for packaging or transporting food. HDPE is used for manufacturing garbage bags, laundry bags, and utility bags, among others. It is also used for manufacturing paper used in handling, packaging or serving food or materials that require a CO2 or oxygen barrier.

Low-Density Polyethylene—LDPE: LDPE plastic bags are commonly used in the manufacture of utility bags and plastics for use in food packaging. The polymer is made of short and branched chains that are of low density and widely spread. This results in a plastic film that is light in weight and of moderately low tensile strength. The film or resulting bag is not stretchable like the HDPE. LDPE comes with a low melting point, making it ideal for heat sealing applications. The light polymer means that the material is easy to see through. It is therefore quite easy to identify the content carried in the paper. LDPE is often used at restaurants and food services for packaged foods. Dry cleaners, butcheries, and suppliers of consumable produce commonly use this type of paper.

Linear Low-Density Polyethylene—LLDPE: LLDPE plastic bags material used in the manufacture of plastic bags for use in newspaper bags, garbage bags, and food bags, among others. The polymer used features non-branching chains but the tensile strength is low compared to that of HDPE. The gauge and clarity of LLDPE are slightly lower compared to high-density polyethylene plastic but the strength is maintained. As a result, they are cost-effective and thus preferred compared to low-density polyethylene plastics. This plastic bag is common with refrigerator storage and laundry stores.

Medium Density Polyethylene MDPE: This is the middle ground between high density and low-density polyethylene. Though it is not as opaque as high density, the film is not as clear as low-density polyethylene. The resulting film is also not dense enough to accommodate bulk storage. MDPE is highly tolerant to chemical encounter. It comes with low tensile properties and easily stretches. It is therefore used in garbage bags and packaging films.

Polypropylene—PP: Polypropylene is one of the most popular types of plastic bags. The material has extremely impressive chemical resistance properties. It is also very strong and can withstand heat because of a high melting point, It is, however, less opaque and will reveal the content of your bag. The longer shelf life, being non-breathable and food safe make the plastic ideal for retail operations. PP generally finds use in packaging and storage of ketchup, candies, syrups, and medicines, among other liquid sensitive items, It can comfortably use for packaging hot supplies without damage.

Other specialty plastics, laminated plastics, co-extruded plastics, mixed plastics, combination woven and extruded plastic type, plastic/paper laminates, filler containing plastics, modified plastics, printed plastics, and other engineer plastics may fine use and refinements for the invention described.

Bag sizes can range from 1 inch squared to over 40 inches squared. More often, bags will be useful from between 2 inch×2 inches squared to 36 inch×36 inches. Most often, convenient bag sizes will range from 6 inch×6 inch to 24 inches×24 inches. Depending on the application, sterilization bags will be longer in length than width or alternatively, wider than longer. Larger bags require pro rata larger volumes of sanitizing agent to satisfy sufficient vaper dispersion and concentration for decontamination.

Bag body mill thickness can range from 0.001 inches thick to 0.125 inches thick depending on the material type and application. More often the thickness range will be from 0.002 inches to 0.1 inches thick. Typically the thickness will range from 0.003 to 0.05 inches. Most often, thickness will be in manufactured range from 0.005 to 0.01 inches thickness. Other components including bag lip, sealing element and handle will have a thickness that will depend on structure, intended weight and application

Consumers can benefit with handles constructed into the back to ensure easy and convenient transport and handling. Bag handles can be incorporated into the sanitary bag during manufacturing or post applied after manufacturing. Likewise, sanitization bags may not need a handle if the sanitizing bag can be placed with an existing bag such as a standard paper bag or re-usable store type bag.

Bags may include a convenient re-sealable zip-lock or zip tie provided that it is easy to seal and unseal the bag. Importantly, FDA grade plastics and components are to be used since articles to be decontaminated during storage and transfer in the bag may food and/or drug products where compliant materials should be utilized.

Temperature indication based on thermochromic pigments can be employed in the plastic of the container to ensure that components contained in the vessel can be kept at a given temperature base on appropriate and safe storage temperatures.

### Sanitizing/Sterilizing Activating Agent Release Constructs:

Generally, a sanitizing/sterilizing activating pouch construct includes, but is not limited to an entrapping barrier, a liquid or otherwise absorbing component for holding and emanating a sanitizing or sterilizing agent, and a secondary apposing entrapping barrier to ensure containment of the sanitizing or sterilizing agent. The sanitizing/sterilizing activating pouch construct combines the ease-of-use for handling, opening for activation, and placement within a sanitizing bag, box, or other decontamination design presented. The construct is intended to contain and not release a sanitizing agent until a seal is intentionally broken upon activation. Activation can be a simple seal break, peel-off mechanism, rupture mechanism, pinch mechanism, actuation, articulation, twist mechanism, or any of a variety of physical or mechanical means for intentional release.

sanitizing/sterilizing activating pouch constructs are comprised using a variety of convenient means for assembly, cost consideration, ease-of-use, safety, and as single-use or re-usable element of the disinfecting system. There are a wide range of plastics, coated papers, film, resins, building materials, foils, metal, metalized films, laminated films, extruded films, cast films, co-extruded films, injection molded plastics, blow-molded plastics, compression molded films, pressure formed films, spray coated films, injection molded sheets, and other relevant processed materials that can assist as elements useful for activating pouch constructs. Tubular constructs such as extruded straws that can be heat-sealed can find use as highly economical barriers sanitizing agents. Likewise, plastic extruded tubes and straws are easily sealed and may be opened or readily as needed for activation. Twist caps, twist lock caps, snap-off caps and the like can be easily injection molded and at high volume for minimizing manufacturing cost so as to benefit both the producer and consumer of the product.

sanitizing/sterilizing activating pouch constructs can be drop-in types or be integrated into a sanitizing chamber. Drop-in types may be small or large depending on the intended volume or release required for an adequate amount of sanitizing agent. Drop-in types can be measure from 1 cm squared to over a 100 cm squared. More often the later dimensions will be from 2 cm squared to 50 cm squared. For convenience and general applications, the drop-in type will from 3 to 10 cm squared. Thickness for drop-in types will range from 1 mm to 50 mm. More often thickness of constructs will be from 2 mm to 10 mm. The lateral size and thickness will depend on the load requirements for a particular product application and they type of sanitizing agent employed.

### Decontamination Indicators for Detecting and Indicating of Sensitizing Release:

Color change indicators can be utilized to indicate the release of a vapor phase disinfectant. By way of example, and not limitation, labels can be printed with an ink comprising a solvent sensitive weakly encapsulated color formers (electron acceptor) combined with separately with weakly encapsulated color developers (electron donor). Labels can be a plastic, paper, film or other practical substrate to create a color change indicator for indicating the presence of a sensitizing decontaminating agent in the vapor phase. The labels can be printed by standard printing processes. When a color change decontamination indicator sensitive to a decontaminating agent is placed onto a decontaminating chamber and an activating sanitizing agent construct is activated and likewise placed into the decontaminating chamber and the chamber sealed, the color change indicator will noticeably change color over time due to the interaction of the sensitizing agent present in the vapor phase.

The color change indicator can be produced to have a time dependent profile that can trigger a color change from a minute to over a 24-hour period or more. Long duration contamination indicators can be used for long term decontamination. Likewise, short term duration contamination indicators can be used for short term decontamination sterilizing agent exposure. The color change chemistry can be adjusted to a corresponding expose-time based composition.

### Materials for Containment of Sensitizing Release Constructs and Chambers:

Materials used for constructs include, but are not limited to a wide range of plastics, sealable papers, metalized barrier films, rubbers, latex, neoprene, polymers, vulcanized materials, thermoset polymers, epoxies, natural rubber, synthetic rubber, bio-degradable plastics, recycled plastics, poly-lactic acid based plastics an related environmentally friendly plastics, plastics with fillers, gel-cap materials, melamine based materials, printable film adhesive sealed, UV adhesive sealed, silicone release coated for easy removal from an adhesive or heat-sealed backing, flexible packaging compositions, and the like depending on the application of interest, material traits for the application, stability of the material with respect to shelf-life and use, ease of manufacturing, component of the construct, compatibility between construct components, cost considerations, and other relevant features. By way of example, not limitation, the bulk of a construct or container may be made with a paper, wood, or natural product for structure where as a barrier using an non-permeable plastic or resin can be incorporated to mitigate solution or gas diffusion as well as provide for an effective seal.

### Sanitizing/Sterilizing Container Constructs:

sanitizing/sterilizing container constructs can be produced in a variety of shapes and sized to meet the needs of an application. Bags, boxes, cylinders, sacks, folders, cases, chests, bottles, jars, travel bags, luggage, shipping containers, crates, vessels, cans, canisters, pods, packages, clam-shells, thermoformed lidded containers and bowls, injection molded containers and lids, bins, hoppers, silos. By way of example, not limitation, a practical and easy to use plastic sealable plastic bag can be used for a variety of consumer, health care, medical, and pharmaceutical goods. To another extreme, large vessels and shipping containers can be used for mass quantities of products to be contained sterilized. Since decontamination and sterilization can be accomplished during transport, time-frames, decontaminating agents, and container designs can be modified accordingly to the application.

Importantly, the container construct should be practical in size and geometry for the intended articles for decontamination. Of equal importance, the container should be sealable during use and practical to open after use. The decontamination chamber should be re-sealable for reusable applications as well as single use applications depending on the application.

### Articles for Decontamination:

Articles intended for decontamination utilizing the invention can come from a wide range of public and private sectors, markets, services, utilities, industries, nationally or globally. Articles are may be any consumer, food-service, health care, residential, hospital, communal, public, private, medical, safety, toy, commercial product, industrial product, component, reading material, clothing item, personal item, essential item, security items, communication devices, protective products, masks, jewelry, clothing, currency, coins paper money, private item, single or multi-use item, valuable or commodity item, packaged or unpackaged item or the like.

Of particular interest are items that may have inadvertently been touched or in contact with a pathogenic source where by the pathogen may contact the item. Practically, any of the above items may risk uncertain contamination including, but not limited to peripheral items that are subject to human, animal, airborne, physical contact or other contact means leading to potential transmission of a pathogen.

By way of example, but not limitation there may be tight limits on availability protective masks, gloves, or other essential health-care protective items. An email to VA medical staff members to encourage healthcare workers to reuse N95 masks and store them in “zip lock bags” in between uses, even though the standard policy is to discard them after they are worn. Decontamination bags discussed herein serve an important, simple and new method to ensure that the masks are not only contained, but also decontaminated during containment.

### Sanitizing/Sterilizing Agents:

Example sanitizing agents include but are not limited to: Benzethonium Chloride CAS # 121-54-0. Primarily as topical antimicrobial agents and preservatives, and secondarily as cationic surfactants. it exhibits a broad spectrum of microbiocidal activity against bacteria, fungi, mold and viruses. Benzalkonium Chloride CAS # 8001-54-5. Primarily used as a preservative and antimicrobial agent, and secondarily used as a surfactant. it works by killing microorganisms and inhibiting their future growth, and for this reason frequently appears as an ingredient in antibacterial hand wipes, antiseptic creams and anti-itch ointments. Benzalkonium Bromide GAS # 7281-04-1. Primarily used as a preservative and antimicrobial agent, and secondarily used as a surfactant. It is effective against gram-positive microbes. Longer expositions may inactivate some viruses. Chlorhexidine Digluconate CAS # 1 8472-51-0. Used in disinfectants (disinfection of the skin and hands), cosmetics (additive to creams, toothpaste, deodorants, and antiperspirants), and pharmaceutical products (preservative in eye drops, active substance in wound dressings and antiseptic mouthwashes). Chlorhexidine Diacetate CAS # 56-95-1. Used primarily as a topical antiseptic/disinfectant in wound healing, at catheterization sites, in various dental applications and in surgical scrubs. It was used to study effective skin antisepsis and disinfection of medical devices as well as the effects of antimicrobials on dental plaque. Chlorhexidine Dihydrochloride CAS # 3697-42-5. An antiseptic antibacterial agent commonly used as a disinfectant in eye drops, deodorants, creams, and others. It is also used as an active ingredient in mouthwash to prevent and control dental plaque. Chlorphenesin CAS # 104-29-0. A synthetic preservative used in skin care as a cosmetic biocide. Most commonly, chlorphenesin is incorporated into cosmetics as a method of preventing bacteria contamination, which may help to prolong product quality and longevity. Polyhexamethyleneguanidine (PHMG)CAS # 57028-96-3. A widely used polymeric antimicrobial agent known to induce significant pulmonary toxicity. It has fungicidal as well as bactericidal activity against both Gram-positive and Gram-negative bacteria. The substance also has detergent, anti-corrosive, and flocculant properties and prevents biofouling. Polyhexamethylenebiguanide (PHMB) CAS # 27083-27-8. A commonly used antiseptic and antimicrobial agent. It is used in a variety of products including wound care dressings, contact lens cleaning solutions, perioperative cleansing products, and swimming pool cleaners, Carbomers (Carbopol) 934, 940, 941, 980CAS # 9007-20-9/76050-42-5, A synthetic high-molecular weight polymer of acrylic acid. Carbomers are thickening agents that help control the viscosity and flow of cosmetic products. They also help distribute and suspend insoluble solids into liquid, and prevent the oil and liquid parts of a solution from separating. This ingredient is contained in a wide range of personal care products such as styling gel, facial moisturizer, sunscreen, shampoo, anti-aging treatment, eye cream, cleanser and scrubs.

Other examples that can be used alone or in combination compounds with antipathogen disinfection capabilities include, but are not limited to: alcohol types such as methanol, ethanol, isopropanol; chloride types such as: sodium hypochlorite, sodium dichloro-s triazinetrione, sodium chlorite, hypochlorous acid, sodium dischloroisocyanurate; formaldehyde;

glutaraldehyde; peroxide types such as: hydrogen peroxide, peracetic acid, peryoxyacetic acid, peroxyoctanoic acid, sodium carbonate peroxyhdrate; phenolic types; combinations of peracetic acid and hydrogen peroxide; ammonium compounds such as: ammonia, quaternary ammonium compounds, ammonium monocarbanate, ammonium bicarbonate; ether type compounds including triethylene glycol; ester type compounds including glycolic acid; and other examples include: silver ionic compounds, citric acid, L-lactic acid and the like.

Formaldehyde and like-kind compounds are used as commercial biocides, preservatives, and in commercial manufacturing processes. Formaldehyde is naturally occurring in many foods consumed and is also produced via common biochemical pathways. It is used in plastics, building materials such as plywood, glues and fabrics, and many house-hold products including medicines and health and beauty aids. Its use should be limited as it is considered a carcinogen. If utilized and limited exposure is involved, it may have beneficial properties such as being an active agent and biocide. It will actively kill all pathogens when exposed to said pathogens. Formaldehyde has been used in vaccines to detoxify bacterial toxins (i.e., diphtheria toxin, tetanus toxin, pertussis toxins) and to inactivate viruses (i.e., poliovirus). Because formaldehyde at high concentrations can cause mutational changes in cellular DNA in vitro, some parents have become concerned that formaldehyde in vaccines might be dangerous. However, because formaldehyde is a product of single-carbon metabolism, everyone has formaldehyde that is detectable in serum. Indeed, the level of formaldehyde in the circulation is approximately 10-fold more than would be contained in any vaccine. Also, people exposed to high levels of formaldehyde in the workplace (e.g., morticians) are not at greater risk of cancer than people who are not exposed to formaldehyde. Finally, the quantity of formaldehyde present in vaccines is at least 600-fold lower than that necessary to induce toxicity in experimental animals. When presently and according to federal guidelines, formaldehyde can be an effective kill agent for surface bound viruses, bacteria and spores. It can be particularly useful when it is limited to killing surface borne pathogens, but subsequently mitigated from ingestion or inhalation.

### Gas Phase Disinfectants:

Ozone is a gas used for disinfecting water, laundry, foods, air, and surfaces. It is chemically aggressive and destroys many organic compounds, resulting in rapid decolorization and deodorization in addition to disinfection. Ozone decomposes relatively quickly. However, due to this characteristic of ozone, tap water chlorination cannot be entirely replaced by ozonation, as the ozone would decompose already in the water piping. Instead, it is used to remove the bulk of oxidizable matter from the water, which would produce small amounts of organochlorides if treated with chlorine only. Regardless, ozone has a very wide range of applications from municipal to industrial water treatment due to its powerful reactivity.

The surface disinfectant effect of glutaraldehyde in the gas-aerosol phase was investigated at different relative humidity and temperatures. At a gas-aerosol concentration of 15 to 20 mg/m3 and a relative humidity of about 80%, glutaraldehyde had a good disinfectant effect against both vegetative bacteria (decimal reduction time, less than 5 min) and bacterial spores (decimal reduction time, less than 45 min). In spite of its low volatility, glutaraldehyde was more effective than formaldehyde when the two substances were compared on an “added amount” basis.

Alkylating agents such as formaldehyde, ethylene oxide, and propylene oxide are broad-spectrum biocides active against bacteria, viruses, and fungi, including spores. Ethylene and propylene oxides are highly reactive gaseous fumigants used to sterilize animal feed, human food, surgical equipment that cannot be autoclaved (e.g., endoscopes, gloves, syringes, catheters, tubing, implantable devices), laboratory equipment, etc. Both are noncorrosive. However, ethylene oxide has better penetrability than propylene oxide and, therefore, is more commonly used. For this application, ethylene oxide is mixed with chlorofluorocarbons or carbon dioxide and sold in gas cylinders.

Antiseptics and disinfectants are extensively used in hospitals and other health care settings for a variety of topical and hard-surface applications. A wide variety of active chemical agents (biocides) are found in these products, many of which have been used for hundreds of years, including alcohols, phenols, iodine, and chlorine. Most of these active agents demonstrate broad-spectrum antimicrobial activity; however, little is known about the mode of action of these agents in comparison to antibiotics. This review considers what is known about the mode of action and spectrum of activity of antiseptics and disinfectants. The widespread use of these products has prompted some speculation on the development of microbial resistance, in particular whether antibiotic resistance is induced by antiseptics or disinfectants.

Many heat-sensitive medical devices and surgical supplies can be effectively sterilized by liquid sterilants (in particular glutaraldehyde, PAA, and hydrogen peroxide) or by vapor-phase sterilization systems. The most widely used active agents in these “cold” systems are ethylene oxide, formaldehyde and, more recently developed, hydrogen peroxide and PAA. Ethylene oxide and formaldehyde are both broad-spectrum alkylating agents. However, their activity is dependent on active concentration, temperature, duration of exposure, and relative humidity. As alkylating agents, they attack proteins, nucleic acids, and other organic compounds; both are particularly reactive with sulfhydryl and other enzyme-reactive groups. Ethylene oxide gas has the disadvantages of being mutagenic and explosive but is not generally harsh on sensitive equipment, and toxic residuals from the sterilization procedure can be routinely eliminated by correct aeration. Formaldehyde gas is similar and has the added advantage of being nonexplosive but is not widely used in health care. Vapor-phase hydrogen peroxide and PAA are considered more active (as oxidants) at lower concentrations than in the liquid form. Both active agents are used in combination with gas plasma in low-temperature sterilization systems. Their main advantages over other vapor-phase systems include low toxicity, rapid action, and activity at lower temperature; the disadvantages include limited penetrability and applications.

Peracetic acid (PAA) is considered a more potent biocide than hydrogen peroxide, being sporicidal, bactericidal, virulcide, and fungicidal at low concentrations (<0.3%). PAA also decomposes to safe by-products (acetic acid and oxygen) but has the added advantages of being free from decomposition by peroxidases, unlike H₂O₂, and remaining active in the presence of organic loads. Its main application is as a low-temperature liquid sterilant for medical devices, flexible scopes, and hemodialyzers, but it is also used as an environmental surface sterilant.

Patent U.S. Pat. No. 3,908,031 A describes a method tor sterilizing materials which do not lend themselves to sterilization with high temperatures and/or steam, and particularly food and food products, wherein the material to be sterilized is contacted with ethanol in the vapor phase, The invention describes an industrial method for the sterilization of materials which do not lend themselves to sterilization with heat and/or steam in which the material to be sterilized is contacted with ethanol in the vapor phase. It found that ethanol vapor is more effective as a sterilizing medium than heat or ethanol in the liquid phase.

The CDC previously announced that a previously unrecognized human virus from the coronavirus family is the leading infect humans cause one third of common colds and can cause gastroenteritis. The virucidal efficacy of chemical germicides against coronavirus has been investigated. A study of disinfectants against coronavirus 229E found several that were effective after a 1-minute contact time; these included sodium hypochlorite (at a free chlorine concentration of 1,000 ppm and 5,000 ppm), 70% 0.05% benzalkonium chloride, 50 ppm iodine in iodophor, 0.23% sodium chlorite, 1% cresol soap and 0.7% formaldehyde inactivated >3 logs of two animal coronaviruses (mouse hepatitis virus, canine e SARS coronavirus by 70% ethanol and povidone-iodine with an exposure times of 1 minute and 2.5% glutaraldehyde with an temperature for at least 1-2 days (WHO, 2003; 2003_05_04/en/index.html), surfaces might be a possible source of contamination and lead to infection with the SARS coronavirus and should be disinfected.

Volatile solvents including methanol, ethanol, propanol, isopropanol and the like can be used in pure form or with added water to increase internal decontamination chamber humidity for increasing effectiveness. Concentrations can range from 10 percent alcohol to 100 percent. More often concentration ranges will be between 30 percent alcohol to 100 percent. Most often the range will be between 50 percent to 100 percent alcohol has the most compatible range.

### Viral Pathogens to be Considered:

The selection of one or more anti-pathogens will be dependent on the final sterilization chamber configuration, hydration or volatilization solution, water content, solvent content, concentration, azeotropic solvent carriers, co-volatilization compositions, placement in the chamber and the like. Traces of the coronavirus SARS-CoV-2 (responsible for COVID-19 disease) were found on surfaces including in cruise-ship cabins for as many as 17 days after passengers left. Although it is not possible to determine any specific infectious diseases are present on a particular surface, decontaminating those surfaces are critical to best ensure that SARS-CoV-2 was or other pathogens are present. Importantly, reducing the spread, cross-contamination, propagation, surface contact propagation, and transfer of any virulent diseases are of critical importance. Any means to abate the spread of further presence and transfer important during epidemic and/or pandemic contagious events.

Importantly in consideration of the unfortunate pandemic outbreak of SARS-CoV-2, it has become essential to protect against this and other pathogens that in general are communicable and can rapidly spread. The chart table below lists viral pathogens, their genus and family as well as the host that can be infected. Many pathogens can cross over between species. Viral pathogens for consideration include, but are not limited to:

| Virus | Genus, Family | Host |
|---|---|---|
| Adeno-associated | Dependovirus, | Human, |
| virus | Parvoviridae | vertebrates |
| Aichi virus | Kobuvirus, | Human |
| | Picornaviridae | |
| Australian bat | Lyssavirus, | Human, bats |
| lyssavirus | Rhabdoviridae | |
| BK polyomavirus | Polyomavirus, | Human |
| | Polyomaviridae | |
| Banna virus | Seadornavirus, | Human, cattle, |
| | Reoviridae | pig, |
| | | mosquitoes |
| Barmah forest virus | Alphavirus, | Human, |
| | Togaviridae | marsupials, |
| | | mosquitoes |
| Bunyamwera virus | Orthobunyavirus, | Human, |
| | Bunyaviridae | mosquitoes |
| Bunyavirus La Crosse | Orthobunyavirus, | Human, deer, |
| | Bunyaviridae | mosquitoes, |
| | | tamias |
| Bunyavirus snowshoe | Orthobunyavirus, | Human, |
| hare | Bunyaviridae | rodents, |
| | | mosquitoes |
| Cercopithecine | Lymphocryptovirus, | Human, |
| herpesvirus | Herpesviridae | monkeys |
| Chandipura virus | Vesiculovirus, | Human, |
| | Rhabdoviridae | sandflies |
| Chikungunya virus | Alphavirus, | Human, |
| | Togaviridae | monkeys, |
| | | mosquitoes |
| Cosavirus A | Cosavirus, | Human |
| | Picornaviridae | |
| Cowpox virus | Orthopoxvirus, | Human, |
| | Poxviridae | mammals |
| Coxsackievirus | Enterovirus, | Human |
| | Picornaviridae | |
| Crimean-Congo | Nairovirus, | Human, |
| hemorrhagic fever | Bunyaviridae | vertebrates, |
| virus | | ticks |
| Dengue virus | Flavivirus, | Human, |
| | Flaviviridae | mosquitoes |
| Dhori virus | Thogotovirus, | Human, ticks |
| | Orthomyxoviridae | |
| Dugbe virus | Nairovirus, | Human, ticks |
| | Bunyaviridae | |
| Duvenhage virus | Lyssavirus, | Human, |
| | Rhabdoviridae | mammals |
| Eastern equine | Alphavirus, | Human, birds, |
| encephalitis virus | Togaviridae | mosquitoes |
| Ebolavirus | Ebolavirus, | Human, |
| | Filoviridae | monkeys, bats |
| Echovirus | Enterovirus, | Human |
| | Picornaviridae | |
| Encephalomyocarditis | Cardiovirus, | Human, |
| virus | Picornaviridae | mouse, rat, pig |
| Epstein-Barr virus | Lymphocryptovirus, | Human |
| | Herpesviridae | |
| European bat | Lyssavirus, | Human, bats |
| lyssavirus | Rhabdovirus | |
| GB virus C/Hepatitis | Pegivirus, | Human |
| G virus | Flaviviridae | |
| Hantaan virus | Hantavirus, | Human, |
| | Bunyaviridae | rodents |
| Hendra virus | Henipavirus, | Human, horse, |
| | paramyxoviridae | bats |
| Hepatitis A virus | Hepatovirus, | Human |
| | picornaviridae | |
| Hepatitis B virus | Orthohepadnavirus, | Human, |
| | Hepadnaviridae | Chimpanzees |
| Hepatitis C virus | Hepacivirus, | Human |
| | Flaviviridae | |
| Hepatitis E virus | Hepevirus, | Human, pig, |
| | Unassigned | monkeys, |
| | | some rodents, |
| | | chicken |
| Hepatitis delta virus | Deltavirus, | Human |
| | Unassigned | |
| Horsepox virus | Orthopoxvirus, | Human, horses |
| | Poxviridae | |
| Human adenovirus | Mastadenovirus, | Human |
| | Adenoviridae | |
| Human astrovirus | Mamastrovirus, | Human |
| | Astroviridae | |
| Human coronavirus | Alphacoronavirus, | Human |
| | Coronaviridae | |
| Human | Cytomegalovirus, | Human |
| cytomegalovirus | Herpesviridae | |
| Human enterovirus | Enterovirus, | Human |
| 68, 70 | Picornaviridae | |
| Human herpesvirus 1 | Simplexvirus, | Human |
| | Herpesviridae | |
| Human herpesvirus 2 | Simplexvirus, | Human |
| | Herpesviridae | |
| Human herpesvirus 6 | Roseolovirus, | Human |
| | Herpesviridae | |
| Human herpesvirus 7 | Roseolovirus, | Human |
| | Herpesviridae | |
| Human herpesvirus 8 | Rhadinovirus, | Human |
| | Herpesviridae | |
| Human | Lentivirus, | Human |
| immunodeficiency | Retroviridae | |
| virus | | |
| Human | Mupapillomavirus, | Human |
| papillomavirus 1 | Papillomaviridae | |
| Human | Alphapapillomavirus, | Human |
| papillomavirus 2 | Papillomaviridae | |
| Human | Alphapapillomavirus, | Human |
| papillomavirus 16, 18 | Papillomaviridae | |
| Human parainfluenza | Respirovirus, | Human |
| | Paramyxoviridae | |
| Human parvovirus | Erythrovirus, | Human |
| B19 | Parvoviridae | |
| Human respiratory | Orthopneumovirus, | Human |
| syncytial virus | Pneumoviridae | |
| Human rhinovirus | Enterovirus, | Human |
| | Picornaviridae | |
| Human SARS | Betacoronavirus, | Human, palm |
| coronavirus | Coronaviridae | civet |
| Human spumaretrovirus | Spumavirus, | Human |
| | Retroviridae | |
| Human T-lymphotropic | Deltaretrovirus, | Human |
| virus | Retroviridae | |
| Human torovirus | Torovirus, | Human |
| | Coronaviridae | |
| Influenza A virus | Influenzavirus A, | Human, birds, |
| | Orthomyxoviridae | pigs |
| Influenza B virus | Influenzavirus B, | Human |
| | Orthomyxoviridae | |
| Influenza C virus | Influenzavirus C, | Human |
| | Orthomyxoviridae | |
| Isfahan virus | Vesiculovirus, | Human, |
| | Rhabdoviridae | sandflies, |
| | | gerbils |
| JC polyomavirus | Polyomavirus, | Human |
| | Polyomaviridae | |
| Japanese encephalitis | Flavivirus, | Human, |
| virus | Flaviviridae | horses, birds, |
| | | mosquitoes |
| Junin arenavirus | Arenavirus, | Human, |
| | Arenaviridae | rodents |
| KI Polyomavirus | Polyomavirus, | Human |
| | Polyomaviridae | |
| Kunjin virus | Flavivirus, | Human, |
| | Flaviviridae | horses, birds, |
| | | mosquitoes |
| Lagos bat virus | Lyssavirus, | Human, |
| | Rhabdoviridae | mammals |
| Lake Victoria | Marburgvirus, | Human, |
| marburgvirus | Filoviridae | monkeys, bats |
| Langat virus | Flavivirus, | Human, ticks |
| | Flaviviridae | |
| Lassa virus | Arenavirus, | Human, rats |
| | Arenaviridae | |
| Lordsdale virus | Norovirus, | Human |
| | Caliciviridae | |
| Louping ill virus | Flavivirus, | Human, |
| | Flaviviridae | mammals, |
| | | ticks |
| Lymphocytic | Arenavirus, | Human, |
| choriomeningitis virus | Arenaviridae | rodents |
| Machupo virus | Arenavirus, | Human, |
| | Arenaviridae | monkeys, |
| | | mouse |
| Mayaro virus | Alphavirus, | Human, |
| | Togaviridae | mosquitoes |
| MERS coronavirus | Betacoronavirus, | Human, Tomb |
| | Coronaviridae | bat |
| Measles virus | Morbilivirus, | Human |
| | Paramyxoviridae | |
| Mengo | Cardiovirus, | Human, |
| encephalomyocarditis | Picornaviridae | mouse, rabbit |
| virus | | |
| Merkel cell | Polyomavirus, | Human |
| polyomavirus | Polyomaviridae | |
| Mokola virus | Lyssavirus, | Human, |
| | Rhabdoviridae | rodents, cat, |
| | | dog shrew |
| Molluscum | Molluscipoxvirus, | Human |
| contagiosum virus | Poxviridae | |
| Monkeypox virus | Orthopoxvirus, | Human, |
| | Poxviridae | mouse, prairie |
| | | dog |
| Mumps virus | Rubulavirus, | Human |
| | Paramyxoviridae | |
| Murray valley | Flavivirus, | Human, |
| encephalitis virus | Flaviviridae | mosquitoes |
| New York virus | Hantavirus, | Human, mouse |
| | Bunyavirus | |
| Nipah virus | Henipavirus, | Human, bats |
| | Paramyxoviridae | |
| Norwalk virus | Norovirus, | Human |
| | Caliciviridae | |
| O'nyong-nyong virus | Alphavirus, | Human, |
| | Togaviridae | mosquitoes |
| Orf virus | Parapoxvirus, | Human, |
| | Poxviridae | mammals |
| Oropouche virus | Orthobunyavirus, | Human, wild |
| | Bunyaviridae | animals(sloths) |
| Pichinde virus | Arenavirus, | Human, rat, |
| | Arenaviridae | guinea pig |
| Poliovirus | Enterovirus, | Human, |
| | Picornaviridae | mammals |
| Punta toro | Phlebovirus, | Human, |
| phlebovirus | Bunyaviridae | sandflies |
| Puumala virus | Hantavirus, | Human, bank |
| | Bunyavirus | vole |
| Rabies virus | Lyssavirus, | Human, |
| | Rhabdoviridae | mammals |
| Rift valley fever virus | Phlebovirus, | Human, |
| | Bunyaviridae | mammals, |
| | | mosquitoes, |
| | | sandflies |
| Rosavirus A | Rosavirus, | Human |
| | Picornaviridae | |
| Ross river virus | Alphavirus, | Human, |
| | Togaviridae | mosquitoes, |
| | | marsupials |
| Rotavirus A | Rotavirus, | Human |
| | Reoviridae | |
| Rotavirus B | Rotavirus, | Human |
| | Reoviridae | |
| Rotavirus C | Rotavirus, | Human |
| | Reoviridae | |
| Rubella virus | Rubivirus, | Human |
| | Togaviridae | |
| Sagiyama virus | Alphavirus, | Human, horse, |
| | Togaviridae | pig, |
| | | mosquitoes |
| Salivirus A | Salivirus, | Human |
| | Picornaviridae | |
| Sandfly fever Sicilian | Phlebovirus, | Human, |
| virus | Bunyaviridae | sandflies |
| Sapporo virus | Sapovirus, | Human |
| | Caliciviridae | |
| Semliki forest virus | Alphavirus, | Human, birds, |
| | Togaviridae | hedgehog, |
| | | mosquitoes |
| Seoul virus | Hantavirus, | Human, rats |
| | Bunyavirus | |
| Simian foamy virus | Spumavirus, | Human, |
| | Retroviridae | monkeys |
| Simian virus 5 | Rubulavirus, | Human, dog |
| | Paramyxoviridae | |
| Sindbis virus | Alphavirus, | Human, birds, |
| | Togaviridae | mosquitoes |
| Southampton virus | Norovirus, | Human |
| | Caliciviridae | |
| St. louis encephalitis | Flavivirus, | Human, birds, |
| virus | Flaviviridae | mosquitoes |
| Tick-borne powassan | Flavivirus, | Human, ticks |
| virus | Flaviviridae | |
| Torque teno virus | Alphatorquevirus, | Human |
| | Anelloviridae | |
| Toscana virus | Phlebovirus, | Human, |
| | Bunyaviridae | mosquitoes |
| Uukuniemi virus | Phlebovirus, | Human, ticks |
| | Bunyaviridae | |
| Vaccinia virus | Orthopoxvirus, | Human, |
| | Poxviridae | mammals |
| Varicella-zoster virus | Varicellovirus, | Human |
| | Herpesviridae | |
| Variola virus | Orthopoxvirus, | Human |
| | Poxviridae | |
| Venezuelan equine | Alphavirus, | Human, |
| encephalitis virus | Togaviridae | rodents, |
| | | mosquitoes |
| Vesicular stomatitis | Vesiculovirus, | Human, cattle, |
| virus | Rhabdoviridae | horse, pig, flies |
| Western equine | Alphavirus, | Human, |
| encephalitis virus | Togaviridae | vertebrates, |
| | | mosquitoes |
| WU polyomavirus | Polyomavirus, | Human |
| | Polyomaviridae | |
| West Nile virus | Flavivirus, | Human, birds, |
| | Flaviviridae | ticks, |
| | | mosquitoes |
| Yaba monkey tumor | Orthopoxvirus, | Human, |
| virus | Poxviridae | monkeys |
| Yaba-like disease | Orthopoxvirus, | Human, |
| virus | Poxviridae | monkeys |
| Yellow fever virus | Flavivirus, | Human, |
| | Flaviviridae | monkeys, |
| | | mosquitoes |
| Zika virus | Flavivirus, | |
| | Flaviviridae | |

### Bacterial Pathogens to be Considered:

Further consideration of bacterial contamination is critical under every day, epidemic, and pandemic circumstances. It is likewise essential to protect against bacterial pathogens that in general, are communicable and can rapidly spread. The chart table below lists bacterial pathogens, their genus and family as well as the host that can be infected. Many pathogens can cross over between species. Bacterial pathogens for consideration include, but are not limited to:

| Genus | Species |
|---|---|
| Actinomyces | Actinomyces Israelii |
| Bacillus | Bacillus anthracis |
| | Bacillus cereus |
| Bacteroides | Bacteroides Fragilis |
| Bartonella | Bartonella henselae |
| | Bartonella quintana |
| Bordetella | Bordetella pertussis |
| Borrelia | Borrelia burgdorferi |
| | Borrelia garinii |
| | Borrelia afzelii |
| | Borrelia recurrentis |
| Brucella | Brucella abortus |
| | Brucella canis |
| | Brucella melitensis |
| | Brucella suis |
| Campylobacter | Campylobacter jejuni |
| Chlamydia and Chlamydophila | Chlamydia pneumoniae |
| | Chlamydia trachomatis |
| | Chlamydophila psittaci |
| Clostridium | Clostridium botulinum |
| | Clostridium difficile |
| | Clostridium perfringens |
| | Clostridium tetani |
| Corynebacterium | Corynebacterium diphtheriae |
| Enterococcus | Enterococcus faecalis |
| | Enterococcus faecium |
| Escherichia | Escherichia coli |
| Ehrlichia | Ehrlichia canis |
| | Ehrlichia chaffeensis |
| Enterococcus | Enterococcus faecalis |
| | Enterococcus faecium |
| Francisella | Francisella tularensis |
| Haemophilus | Haemophilus influenzae |
| Helicobacter | Helicobacter pylori |
| Klebsiella | Klebsiella pneumoniae |
| Legionella | Legionella pneumophila |
| Leptospira | Leptospira interrogans |
| | Leptospira santarosai |
| | Leptospira weilii |
| | Leptospira noguchii |
| Listeria | Listeria monocytogenes |
| Mycobacterium | Mycobacterium leprae |
| | Mycobacterium tuberculosis |
| | Mycobacterium ulcerans |
| Mycoplasma | Mycoplasma pneumoniae |
| Neisseria | Neisseria gonorrhoeae |
| | Neisseria meningitidis |
| Pseudomonas | Pseudomonas aeruginosa |
| Rickettsia | Rickettsia rickettsii |
| Salmonella | Salmonella typhi |
| | Salmonella typhimurium |
| Shigella | Shigella sonnei |
| Staphylococcus | Staphylococcus aureus |
| | Staphylococcus epidermidis |
| | Staphylococcus saprophyticus |
| Streptococcus | Streptococcus agalactiae |
| | Streptococcus pneumoniae |
| | Streptococcus pyogenes |
| Treponema | Treponema pallidum |
| Ureaplasma | Ureaplasma urealyticum |
| Vibrio | Vibrio cholerae |
| Yersinia | Yersinia pestis |
| | Yersinia enterocolitica |
| | Yersinia pseudotuberculosis |

### Spread of SARS-CoV-2:

Public health responses to SARS-CoV-2 (COVID-19) outbreaks on cruise ships were aimed at limiting transmission among passengers and crew, preventing exportation of SARS-CoV-2 (COVID-19) to other communities, and assuring the safety of travelers and responders.

These responses required the coordination of stakeholders across multiple sectors, including U.S. Government departments and agencies, foreign ministries of health, foreign embassies, state and local public health departments, hospitals, laboratories, and cruise ship companies. At the time of the Diamond Princess outbreak, it became apparent that passengers disembarking from cruise ships could be a source of community transmission. Therefore, aggressive efforts to contain transmission on board and prevent further transmission upon disembarkation and repatriation were instituted. These efforts included travel restrictions applied to persons, movement restrictions applied to ships, infection prevention and control measures, (e.g., use of personal protective equipment for medical and cleaning staff), disinfection of the cabins of persons with suspected COVID-19, provision of communication materials, notification of state health departments, and investigation of contacts of cases identified among U.S. returned travelers.

Cruise ships are often settings for outbreaks of infectious diseases because of their closed environment, contact between travelers from many countries, and crew transfers between ships. On the Diamond Princess, transmission largely occurred among passengers before quarantine was implemented, whereas crew infections peaked after quarantine. On the Grand Princess, crew members were likely infected on voyage A and then transmitted SARS-CoV-2 to passengers on voyage B. The results of testing of passengers and crew on board the Diamond Princess demonstrated a high proportion (46.5%) of asymptomatic infections at the time of testing. Available statistical models of the Diamond Princess outbreak suggest that 17.9% of infected persons never developed symptoms. A high proportion of asymptomatic infections could partially explain the high attack rate among cruise ship passengers and crew. SARS-CoV-2 RNA was identified on a variety of surfaces in cabins of both symptomatic and asymptomatic infected passengers up to 17 days after cabins were vacated on the Diamond Princess but before disinfection procedures had been conducted (Takuya Yamagishi, National Institute of Infectious Diseases, personal communication, 2020). Although these data cannot be used to determine whether transmission occurred from contaminated surfaces, further study of fomite transmission of SARS-CoV-2 aboard cruise ships is warranted.

During the initial stages of the COVID-19 pandemic, the Diamond Princess was the setting of the largest outbreak outside mainland China. Many other cruise ships have since been implicated in SARS-CoV-2 transmission. Factors that facilitate spread on cruise ships might include mingling of travelers from multiple geographic regions and the closed nature of a cruise ship environment. This is particularly concerning for older passengers, who are at increased risk for serious complications of COVID-19. The Grand Princess was an example of perpetuation of transmission from crew members across multiple consecutive voyages and the potential introduction of the virus to passengers and crew on other ships. Public health responses to cruise ship outbreaks require extensive resources. Temporary suspension of cruise ship travel during the current phase of the COVID-19 pandemic has been partially implemented by cruise lines through voluntary suspensions of operations, and by CDC through its unprecedented use of travel notices and warnings for conveyances to limit disease transmission.

The following examples are offered by way of illustration and not by way of limitation.

### EXAMPLES

### 1. Activating Disinfecting Pouch:

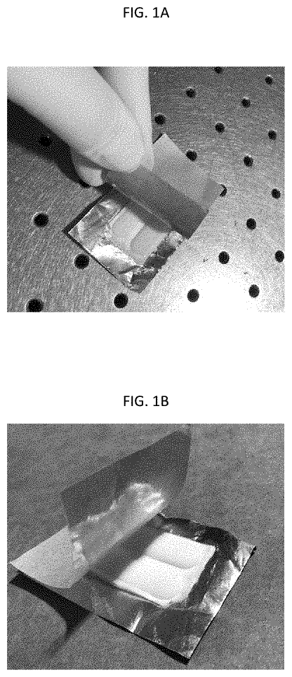

A absorbent pad made of an absorbing cotton strip (or alternatively paper, absorbent and porous to liquid fills) 1.0 inches wide, 0.25 inches thick, and 2.0 inches long is initially laminated on the adhesive side of an adhesive coated foil 1.5 inches wide 0.007 inches thick, and 2.5 inches long. The pad is centered so that at least 0.25 inches of adhesive remains exposed on the aluminum foil barrier. 2.5 ml of a 70% solution of isopropanol and 30% distilled water was applied directly to the absorbing pad. Upon solution application to the absorbent pad, a plastic laminating barrier with a release coating and the same size/orientation as the adhesive coated foil (0.007 inches thick) was applied to the pad/foil pair to hermetically seal the solvent absorbed pad. The laminated construct contains the solution per time required for use. The plastic laminating barrier is readily removed in order to expose the alcohol saturated pad. FIGS. 1A and 1B provide pictures of the construct and removal of the laminating barrier for use. The activating disinfecting pouch can be conveniently stored, activated immediately prior to use and inserted into a decontaminating bag prior to or after placing an article into the back whereby the article is intended for decontamination.

### 2. Pellet Based Activating Disinfecting Pouch:

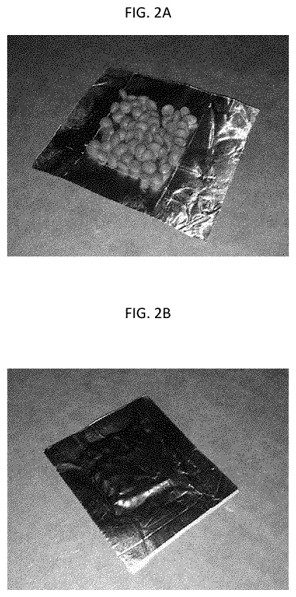

An absorbent pad was made using a master batch pelletizing process. Pellets were infused with low levels of a concentrated volatile water based sterilizing agent described above. The plastic pellets were produced with a polyolefin resin and an extrusion release aid. The infused pellets were initially laminated on the adhesive side of an adhesive coated foil 1.5 inches wide 0.007 inches thick, and 2.5 inches long. The pellets were centered so that at least 0.25 inches of adhesive remains exposed on the aluminum foil barrier. See FIG. 2A. Upon pellet application a plastic laminating barrier with a release coating and the same size/orientation as the adhesive coated foil (0.007 inches thick) is applied to the pellet/foil pair to hermetically seal the infused pellets. See FIG. 2B. The laminated construct contains the solution per time required for use. The plastic laminating barrier is readily removed in order to expose the alcohol saturated pad. The activating disinfecting pellet pouch can be conveniently stored, activated immediately prior to use and inserted into a decontaminating bag prior to or after placing an article into the back whereby the article is intended for decontamination

3. Disinfecting Single-Use Bag used for Plastic Articles:

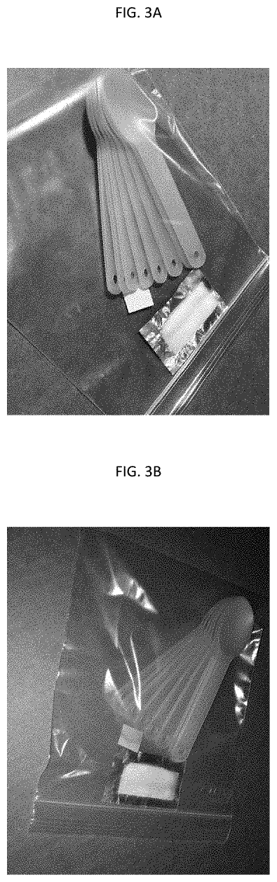

A plastic bag made from polyethylene 0.003 inch thick measuring 6 inches wide by 6 inches in length with an air tight zip seal was utilized as a surface disinfecting bag. An activating disinfecting pouch was produced as described above. The activating disinfecting pouch was activated and placed in the bag. A set of spoons was added to the bag and with proximity of the activated disinfecting pouch. The bag was sealed for 30 minutes at ambient room temperature (68° F.). See FIGS. 3A and 3B. The plastic spoons were removed after 30 minuets exposure time in the disinfecting bag. There was no material or structure changes to the spoons.

4. Disinfecting Single-Use Bag used for N95 Respirator Mask:

A plastic bag made from polyethylene 0.003 inch thick measuring 6 inches wide by 6 inches in length with an air tight zip seal was utilized as a surface disinfecting bag. An activating disinfecting pouch was produced as described above. The activating disinfecting pouch was activated and placed in the bag. A N95 mask was added to the bag and with proximity of the activated disinfecting pouch. The bag was sealed for 60 minutes at ambient room temperature (68° F.). The mask was removed after 60 minuets exposure time in the disinfecting bag. There no material or structure changes to the mask.

5. Disinfecting Multi-Use Bag used for N95 Respirator Mask:

A thicker plastic bag made from polyethylene 0.006 inch thick measuring 6 inches wide by 6 inches in length with an air tight zip seal was utilized as a surface multi-use disinfecting bag. An activating disinfecting pouch was produced as described above. The activating disinfecting pouch was activated and placed in the bag. A N95 mask was added to the bag and with proximity of the activated disinfecting pouch. The bag was sealed for 60 minutes at ambient room temperature (68° F.). The mask was removed after 60 minuets exposure time in the disinfecting bag. There no material or structure changes to the mask.

### 6. Decontamination Indicators for Detecting and Indicating of Sensitizing Release:

A gas phase printed indicator was screen printed. The color change gas sensitive ink was purchased from Segan Industries, Inc., Burlingame Calif. The color change ink was printed under normal screen-printing conditions on to a 0.003 inch thick printable paper substrate. Upon drying air drying and die-cutting down to a 2 cm×2 cm size and then the sensor tabs were stored under dark ambient temperatures until ready for use.

The ink was made from solvent sensitive weakly encapsulated color formers (electron acceptor) combined separately with weakly encapsulated color developers (electron donor). When a color change decontamination indicator sensitive to a decontaminating agent is placed onto a decontaminating chamber and an activating sanitizing agent construct is activated and likewise placed into the decontaminating chamber and the chamber sealed, the color change indicator will noticeably change color over time due to the interaction of the sensitizing agent present in the vapor phase.

### 7. Disinfecting Bag Including Plastic Articles and a Decontamination Indicator:

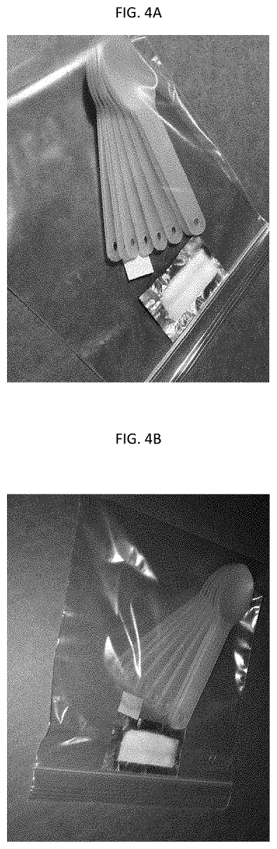

A plastic bag made from polyethylene 0.003 inch thick measuring 6 inches wide by 6 inches in length with an air tight zip seal was utilized as a surface disinfecting bag. An activating disinfecting pouch was produced as described above. A decontamination sensor described above printed for 60 minute indication to a light blue color. The activating disinfecting pouch was activated and placed in the bag. A set of spoons was added to the bag and with proximity of the activated disinfecting pouch. The bag was sealed for 60 minutes at ambient room temperature (68° F.). See FIG. 4A. The decontamination indicator turned to a blue color within the expected 60 minute period confirming the presence of the volatile sterilizing agent. See FIG. 4B. The plastic spoons were removed after 60 minuets exposure time in the disinfecting bag. There were no material or structure changes to the spoons.

### 8. Semi-Mobile Disinfection Chamber:

The outermost part of the chamber is a 24 inches wide×18 inches deep×12 inches container with a detachable lid. The material of the chamber can withstand deterioration from sterilants and high-level disinfectants, it may be composed of polycarbonate and/or acrylonitrile butadiene styrene. A container 4 inches from the bottom of the box is a rack of similar composition that products are placed on. The very bottom of the box has an opening in which a detachable cartridge of disinfectants can be inserted. The cartridge remains sealed until the product user inserts products to be disinfected into the chamber and places the lid. The seal on the bottom of the box can be removed as a pull tab, allowing for vapor to penetrate into the chamber. The amount of time for disinfection may vary.

### 9. Mobile Medium Volume Disinfection Chamber:

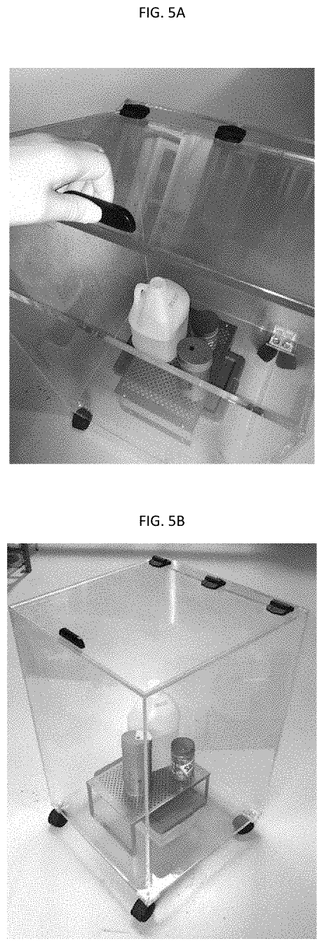

A mobile disinfection chamber 18 inches wide, 18 inches deep and 24 inches tall with a hinged lid was produced from polycarbonate and modified with casters. The material of the chamber can withstand deterioration from sterilants and high-level disinfectants, it may be composed of polycarbonate and/or acrylonitrile butadiene styrene. 4″ from the bottom of the container is a rack of similar composition that products are placed on. The very bottom of the box housed a removable cartridge for disinfectants. The cartridge should remain sealed until the product user inserts products to be disinfected into the chamber and places the lid. The seal on the bottom of the box can be removed as a pull tab, allowing for vapor to penetrate into the chamber. See FIGS. 5A and 5B. The amount of time for disinfection may vary.

Additional details regarding more embodiments of the invention may be found in provisional application Ser. No. 62/994,743, the disclosure of which is herein incorporated by reference.

Aspects of the invention involve a sealed container (e.g. plastic bag or the like), that will include products purchased from a retail, on-line store, commercial and/or industrial companies, and is a simple/safe means to kill viral and bacterial contagions that may in any way be associated with products purchased and intended for the public, private, or elected sector distribution. The invention describes simple, affordable, sustainable, and safe decontamination elements that combined with a hermetically sealed container or otherwise means that will efficiently decontaminate a product received from a supplier intended for a receiver. The means include, but is not limited to, a container/decontaminating element that saturates the container and articles contained within the container. The decontamination element within the container saturates the container at a level of an agent that kills contagions to beyond an acceptable limit to keep those safe.

### 10. Disinfectants for Use in Embodiments of the Invention

Ethanol/Ethyl alcohol
Isopropyl
Superoxidized water
Hydrogen Peroxide
Ammonium carbonate/ammonium bicarbonate combo
Peroxyacetic Acid
Hydrogen Peroxide/Peroxyacetic Acid
Quanternary Ammonium
   Triethylene combo
   Isoproponal combo
   Ethanol combo
   Ethanol/isoproponal combo
Octanoic Acid
Sodium Hypochlorite
   Sodium carbonate combo
Sodium Chlorite
   Sodium Dischloroisocyanurate
Phenolic

### 11. Workplace

Sanitize incoming materials and outgoing products in manufacturing setting. Reduces waist of spraying and wiping. Reduces exposure from lifting, moving and wiping in-coming and out-going products.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A sanitizing device, the device comprising:
a container; and
a sanitizer configured to contact an object present in the container with a sanitizing amount of a volatile antimicrobial composition.

2. The sanitizing device according to claim 1, wherein the antimicrobial composition is anti-viral and/or anti-bacterial.

3. The sanitizing device according to claim 2, wherein the antimicrobial composition comprises an alcohol.

4. The sanitizing device according to claim 3, wherein the alcohol is present in an absorbent pad.

5. The sanitizing device according to claim 1, wherein the container is a bag.

6. The sanitizing device according to claim 5, wherein the bag is a polymeric bag.

7. The sanitizing device according to claim 6, wherein the polymeric bag is sealable.

8. The sanitizing device according to claim 7, wherein the polymeric bag comprises a zip lock seal.

9. The sanitizing device according to claim 1, wherein container includes an object to be sanitized present therein.

10. The sanitizing device according to claim 9, wherein the object is a consumer product.

11. A method of sanitizing an object, the method comprising:
placing the object in a sanitizing device comprising:
a container; and
a sanitizer configured to contact the object present in the container with a sanitizing amount of a volatile antimicrobial composition to sanitize the object; and removing the sanitized object from the container.

12. The method according to claim 1, wherein the antimicrobial composition is anti-viral and/or anti-bacterial.

13. The method according to claim 2, wherein the antimicrobial composition comprises an alcohol.

14. The method according to claim 3, wherein the alcohol is present in an absorbent pad.

15. The method according to claim 1, wherein the container is a bag.

16. The method according to claim 5, wherein the bag is a polymeric bag.

17. The method according to claim 6, wherein the polymeric bag is sealable.

18. The method according to claim 7, wherein the polymeric bag comprises a zip lock seal.

19. The method according to claim 1, wherein container includes an object to be sanitized present therein.

20. The method according to claim 9, wherein the object is a consumer product.
